SEQUENCE LISTING (8 sequences)

SEQ ID NO: 1 - DNA, 1800 bp, Homo sapiens
  CDS (34)..(1758)
gatagtgctgaagaggaggggcgttcccagaccatggcatctacggaaggtgccaacaatatgcccaagcaggtggaagtgcgaatgccagacagtcatcttggctcagaggaacccaagcaccggcacctgggcctgcgcctgtgtgacaagctggggaagaatctgctgctcaccctgacggtgtttggtgtcatcctgggagcagtgtgtggagggcttcttcgcttggcatctcccatccaccctgatgtggttatgttaatagccttcccaggggatatactcatgaggatgctaaaaatgctcattctccctctaatcatctccagcttaatcacagggttgtcaggcctggatgctaaggctagtggccgcttgggcacgagagccatggtgtattacatgtccacgaccatcattgctgcagtactgggggtcattctggtcttggctatccatccaggcaatcccaagctcaagaagcagctggggcctgggaagaagaatgatgaagtgtccagcctggatgccttcctggaccttattcgaaatctcttccctgaaaaccttgtccaagcctgctttcaacagattcaaacagtgacgaagaaagtcctggttgcaccaccgccagacgaggaggccaacgcaaccagcgctgaagtctctctgttgaacgagactgtgactgaggtgccggaggagactaagatggttatcaagaagggcctggagttcaaggatgggatgaacgtcttaggtctgatagggtttttcattgcttttggcatcgctatggggaagatgggagatcaggccaagctgatggtggatttcttcaacattttgaatgagattgtaatgaagttagtgatcatgatcatgtggtactctcccctgggtatcgcctgcctgatctgtggaaagatcattgcaatcaaggacttagaagtggttgctaggcaactggggatgtacatggtaacagtgatcataggcctcatcatccacgggggcatctttctccccttgatttactttgtagtgaccaggaaaaaccccttctccctttttgctggcattttccaagcttggatcactgccctgggcaccgcttccagtgctggaactttgcctgtcacctttcgttgcctggaagaaaatctggggattgataagcgtgtgactagattcgtccttcctgttggagcaaccattaacatggatggtacagccctttatgaagcggtggccgccatctttatagcccaaatgaatggtgttgtcctggatggaggacagattgtgactgtaagcctcacagccaccctggcaagcgtcggcgcggccagtatccccagtgccgggctggtcaccatgctcctcattctgacagccgtgggcctgccaacagaggacatcagcttgctggtggctgtggactggctgctggacaggatgagaacttcagtcaatgttgtgggtgactcttttggggctgggatagtctatcacctctccaagtctgagctggataccattgactcccagcatcgagtgcatgaagatattgaaatgaccaagactcaatccatttatgatgacatgaagaaccacagggaaagcaactctaatcaatgtgtctatgctgcacacaactctgtcatagtagatgaatgcaaggtaactctggcagccaatggaaagtcagccgactgcagtgttgaggaagaaccttggaaacgtgagaaataaggatatgagtctcagcaaattcttgaataaactccccagcgt

SEQ ID NO: 2 - protein, 574 aa, Homo sapiens
MASTEGANNMPKQVEVRMPDSHLGSEEPKHRHLGLRLCDKLGKNLLLTLTVFGVILGAVCGGLLRLASPIHPDVVMLIAFPGDILMRMLKMLILPLIISSLITGLSGLDAKASGRLGTRAMVYYMSTTIIAAVLGVILVLAIHPGNPKLKKQLGPGKKNDEVSSLDAFLDLIRNLFPENLVQACFQQIQTVTKKVLVAPPPDEEANATSAEVSLLNETVTEVPEETKMVIKKGLEFKDGMNVLGLIGFFIAFGIAMGKMGDQAKLMVDFFNILNEIVMKLVIMIMWYSPLGIACLICGKIIAIKDLEVVARQLGMYMVTVIIGLIIHGGIFLPLIYFVVTRKNPFSLFAGIFQAWITALGTASSAGTLPVTFRCLEENLGIDKRVTRFVLPVGATINMDGTALYEAVAAIFIAQMNGVVLDGGQIVTVSLTATLASVGAASIPSAGLVTMLLILTAVGLPTEDISLLVAVDWLLDRMRTSVNVVGDSFGAGIVYHLSKSELDTIDSQHRVHEDIEMTKTQSIYDDMKNHRESNSNQCVYAAHNSVIVDECKVTLAANGKSADCSVEEEPWKREK

SEQ ID NO: 3 - DNA, 20 bp, adeno-associated virus 2
actccatcactaggggttcc

SEQ ID NO: 4 - DNA, 30 bp, adeno-associated virus 2
ggtaatgattaacccgccatgctacttatc

SEQ ID NO: 5 - DNA, 22 bp, adeno-associated virus 2
tcagaatctggcggcaactccc

SEQ ID NO: 6 - DNA, 20 bp, adeno-associated virus 2
tcgtcaaaaaggcgtatcag

SEQ ID NO: 7 - DNA, 20 bp, adeno-associated virus 2
tcccttgtcgagtccgttga

SEQ ID NO: 8 - DNA, 20 bp, adeno-associated virus 2
cagaaggaaaacagcaaacg

(19) United States
(12) Patent Application Publication
(10) Pub. No.: US 2002/0099025 A1
(43) Pub. Date: Jul. 25, 2002

(54) Treatment of neurological disorders

(76) Inventor: James A. Heywood, Newton, MA (US)

Correspondence Address: NUTTER MCCLENNEN & FISH LLP, ONE INTERNATIONAL PLACE, BOSTON, MA, 02110, US

(21) Appl. No.: 09/748,657

(22) Filed: Dec. 22, 2000

Related U.S. Application Data
(60) Provisional application No. 60/173,764, filed on Dec. 30, 1999.

(51) Int. Cl. A61K 48/00; C12N 7/00; C12N 15/861; C12N 15/867

(52) U.S. Cl. 514/44; 424/93.21; 435/456; 435/235.1

(57) ABSTRACT

The invention provides for the use of recombinant AAV virions to deliver an AAV vector containing an EAAT gene such as EAAT2, to provide a therapeutic effect in neurological disorders such as ALS and epilepsy. The invention also provides for non-viral delivery systems to delivery EAAT2 for a therapeutic effect in ALS and epilepsy.

### BACKGROUND OF THE INVENTION

Selective neuron loss during various neurological disorders has been linked to the actions of neurotransmitters such as glutamate and aspartate. These neurological disorders may be a consequence of the mechanisms of neurotransmitter trafficking and appear to involve an increase (e.g., Huntington's Disease, epilepsy), or a decrease (e.g., hypoxia-ischemia and hypoglycemia) in energy demands. Energy depletion enhances both calcium-dependent and independent glutamate release (Drejer et al. (1985) J. Neurochem. 34:145-151 and Dagani et al. (1987) J. Neurochem. 49:1229-1240). Furthermore, energy failure impairs high-affinity glutamate re-uptake into neurons (Drejer et al., supra), which enhances glutamate neurotoxicity (Kohler, et al. (1981) Brain Res. 211:485-491; Choi (1987) J. Neurosci. 7:357-368). Glutamate uptake into glia is energy-dependent (Barbour, et al., (1988) Nature 335:433), and diminution of the glial component of glutamate uptake enhances toxicity.

One such disorder that involves glutamate is amyotrophic lateral sclerosis (ALS), which is a progressive, degenerative disease of the voluntary motor system (See e.g., L P Rowland, Merritt's Textbook of Neurology, ed. L P Rowland, Hereditary and acquired motor neuron disease (Philadelphia: Williams and Wilkins, 1995)). Symptoms of weakness and muscle atrophy usually begin asymmetrically and distally in one limb, and then spread within the neuroaxis to involve contiguous groups of motor neurons. Symptoms can begin either in bulbar or limb muscles. Clinical signs of both lower and upper motor neuron involvement are required for a definitive diagnosis of ALS. Respiration is usually affected late in limb onset patients, but occasionally can be an early manifestation in patients with bulbar onset symptoms. The course of the disease is characterized by a decline in strength typically leading to death within 2-5 years.

Although the etiology of the disease is unknown, the dominant theory is that neuronal cell death in ALS is the result of over-excitement of neuronal cells due to excess extracellular glutamate. Glutamate is a neurotransmitter that is released by glutaminergic neurons, and is taken up into glial cells where it is converted into glutamine by the enzyme glutamine synthetase, glutamine then re-enters the neurons and is hydrolyzed by glutaminase to form glutamate, thus replenishing the neurotransmitter pool. In a normal spinal cord and brain stem, the level of extracellular glutamate is kept at low micromolar levels in the extracellular fluid because glial cells, which function in part to support neurons, use the excitatory amino acid transporter type 2 (EAAT2) protein to absorb glutamate immediately. A deficiency in the normal EAAT2 protein in patients with ALS, was identified as being important in the pathology of the disease (See e.g., Meyer et al., (1998) J. Neurol. Neurosurg Psychiatry, 65:594-596; Aoki et al., (1998) Ann. Neurol 43:645-653; Bristol et al., (1996) Ann Neurol. 39:676-679). One explanation for the reduced levels of EAAT2 is that EAAT2 is spliced aberrantly (Lin et al. (1998) Neuron, 20:589-602). The aberrant splicing produces a splice variant with a deletion of 45 to 107 amino acids located in the C-terminal region of the EAAT2 protein (Meyer et al. (1998) Neureosci Lett. 241:68-70). Due to the lack of, or defectiveness of EAAT2, extracellular glutamate accumulates, causing neurons to fire continuously. The accumulation of glutamate has a toxic effect on neuronal cells because continual firing of the neurons leads to early cell death.

To date, attempts to treat ALS have involved treating neuronal degeneration with long-chain fatty alcohols which have cytoprotective effects (See U.S. Pat. No. 5,135,956); or with a salt of pyruvic acid (See U.S. Pat. No. 5,395,822); and using a glutamine synthetase to block the glutamate cascade (See U.S. Pat. No. 5,906,976). For example, Riluzole™, a glutamate release inhibitor, has been approved in the U.S. for the treatment of ALS, and appears to extend the life of at least some patients with ALS. However, some reports have indicated that even though Riluzole™ therapy can prolong survival time, it does not appear to provide an improvement of muscular strength in the patients. Therefore, the effect of Riluzole™ is limited in that the therapy does not modify the quality of life for the patient (Borras-Blasco et al. (1998) Rev. Neurol., 27:1021-1027).

Accordingly, a need exists to develop therapies that not only prolongs the survival time of patients with ALS, but also improves the quality of their lives. A need exists to use gene therapy to replace the EAAT2 protein in patients with ALS for long term expression of the EAAT2 protein.

### SUMMARY OF THE INVENTION

The present invention provides methods for treating disorders (e.g., ALS, epilepsy, Huntington's Disease and Parkinson's Disease) associated with abberant excitatory amino acid transporter type (EAAT) protein levels in a subject by providing an alteration in the expression of EAAT levels in the subject. These disorders involve excitatory amino acid neurotransmitters and energy depletion that enhances both calcium-dependent and independent glutamate release (Drejer et al. (1985) J. Neurochem. 34:145-151 and Dagani et al. (1987) J. Neurochem. 49:1229-1240).

In particular, the invention provides an approach to circumvent the present limitations of ALS treatment by using gene therapy to replace the genes responsible for the production of the excitatory amino acid transporter type 2 (EAAT2) protein in patients with ALS. The method of the invention provides long-term expression of the EAAT2 protein. Expression of the EAAT2 protein results in the prevention of glutamate toxicity and significantly slows or arrest the progress of the disease.

Accordingly, in one aspect, the invention provides a method for treating a subject with a disorder associated with aberrant production of an excitatory amino acid transporter (EAAT) protein comprising:

transducing mammalian cells with a therapeutically effective amount of a recombinant virus which has encapsidated therein a gene transfer vector comprising:

(i) a first viral inverted terminal repeat sequence; (ii) a nucleotide sequence encoding an EAAT protein operably linked to a promoter functional in stem cells; (iii) a second viral inverted terminal repeat, wherein at least one of the elements (i) and (iii) comprises an AAV packaging signal; and

expressing the EAAT protein at levels that ameliorate the disorder associated with aberrant concentrations of an EAAT protein.

In one embodiment, the step of transducing mammalian cells further comprises transducing mammalian cells with a recombinant virus selected from the group consisting of adenovirus, adeno-associated virus, retrovirus and lentivirus.

In one embodiment, the step of transducing mammalian cells further comprises transducing mammalian cells selected from the group consisting of hematopoietic stem cells, myeloid stem cells, glial cells, cord blood cells, fetal stem cells, pig stem cells, and neuronal cells. The step of transducing mammalian cells further comprises transducing the mammalian cells ex vivo or in vivo.

In another aspect, the invention provides a method for treating a subject with a disorder associated with aberrant production of an excitatory amino acid transporter (EAAT) protein comprising:

transducing stem cells with a therapeutically effective amount of a recombinant adeno-associated virus (AAV) virion which has encapsidated therein a gene transfer vector comprising:

(i) a first AAV inverted terminal repeat sequence; (ii) a nucleotide sequence encoding an EAAT protein operably linked to a promoter functional in stem cells; (iii) a second AAV inverted terminal repeat, wherein at least one of the elements (i) and (iii) comprises an AAV packaging signal; and

expressing the EAAT protein at levels that ameliorate the disorder associated with aberrant concentrations of an EAAT protein.

In a preferred embodiment, the step of transducing stem cells further comprises transducing myeloid stem cells. The step of transducing stem cells further comprises transducing the stem cells ex vivo or in vivo.

In one embodiment, the step of transducing stem cells further comprises transducing stem cells with an AAV virion which has encapsidated therein a gene transfer vector comprising a nucleotide sequence encoding an EAAT protein selected from the group consisting of EAAT1, EAAT2, EAAT3, EAAT4, and EAAT5.

In one embodiment, the promoter is selected from the group consisting of early cytomegalovirus promoter (CMV), herpesvirus thymidine kinase (TK) promoter and CSF-1 promoter.

In yet another aspect, the invention provides a method for treating amyotrophic lateral sclerosis (ALS) comprising:

transducing stem cells with a therapeutically effective amount of a recombinant adeno-associated virus (AAV) particle which has encapsidated therein a gene transfer vector comprising:

(i) a first AAV inverted terminal repeat sequence; (ii) a nucleotide sequence encoding EAAT2 operably linked to a promoter functional in stem cells, (iii) a second AAV inverted terminal repeat, wherein at least one of the elements (i) and (iii) comprises an AAV packaging signal; and

expressing the EAAT2 protein at levels that ameliorate ALS.

In yet another aspect, the invention provides a method for treating amyotrophic lateral sclerosis (ALS) comprising:

transducing glial cells with a therapeutically effective amount of a recombinant adeno-associated virus (AAV) particle which has encapsidated therein a gene transfer vector comprising:

(i) a first AAV inverted terminal repeat sequence; (ii) a nucleotide sequence encoding EAAT2 operably linked to a promoter functional in glial cells, (iii) a second AAV inverted terminal repeat, wherein at least one of the elements (i) and (iii) comprises an AAV packaging signal; and

expressing the EAAT2 protein at levels that ameliorate ALS.

In a preferred embodiment, the promoter is selected from the group consisting of human glial fibrillary acidic protein promoter (GFAP) and rat neuron specific enolase (NSE).

In yet another aspect, the invention provides a method for treating amyotrophic lateral sclerosis (ALS) comprising:

delivering the nucleotide sequence encoding EAAT2 operably linked to a promoter functional to glial cells; and

expressing the EAAT2 protein a levels that ameliorate ALS.

In one embodiment, the step of delivering the nucleotide sequence encoding EAAT2 comprises delivering the nucleotide sequence encoding EAAT2 as a lipid entrapped sequence. In one embodiment, the lipid is selected from the group consisting of egg phosphatidylcholine, dipalmitoylphosphatidylcholine, distearoylphosphatidylcholine, polylysine, protamine, sulfate and 3b-[N-(N′,N′dimethylaminoethane) carbamoyl] cholesterol.

In yet another aspect, the invention provides a method for treating epilepsy comprising:

transducing glial cells with a therapeutically effective amount of a recombinant adeno-associated virus (AAV) virion which has encapsidated therein a gene transfer vector comprising:

(i) a first AAV inverted terminal repeat sequence;

(ii) a nucleotide sequence encoding EAAT2 operably linked to a promoter functional in glial cells,

(iii) a second AAV inverted terminal repeat, wherein at least one of the elements (i) and (iii) comprises an AAV packaging signal; and

expressing the EAAT2 protein at levels that ameliorate epilepsy.

In yet another aspect, the invention provides a method for treating Huntington's disease comprising:

transducing glial cells with a therapeutically effective amount of a recombinant adeno-associated virus (AAV) virion which has encapsidated therein a gene transfer vector comprising:

(i) a first AAV inverted terminal repeat sequence;

(ii) a nucleotide sequence encoding EAAT2 operably linked to a promoter functional in glial cells,

(iii) a second AAV inverted terminal repeat, wherein at least one of the elements (i) and (iii) comprises an AAV packaging signal; and

expressing the EAAT2 protein at levels that ameliorate Huntington's disease.

In yet another aspect, the invention provides a method for treating Parkinson's disease comprising:

transducing glial cells with a therapeutically effective amount of a recombinant adeno-associated virus (AAV) virion which has encapsidated therein a gene transfer vector comprising:

(i) a first AAV inverted terminal repeat sequence;

(ii) a nucleotide sequence encoding EAAT2 operably linked to a promoter functional in glial cells,

(iii) a second AAV inverted terminal repeat, wherein at least one of the elements (i) and (iii) comprises an AAV packaging signal; and

expressing the EAAT2 protein at levels that ameliorate Parkinson's disease.

### DETAILED DESCRIPTION

The practice of the present invention employs, unless otherwise indicated, conventional methods of virology, microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, Vol. I & II (B. N. Fields and D. M. Knipe, eds.))

So that the invention is more clearly understood, the following terms are defined:

The term “gene transfer” or “gene delivery” as used herein refers to methods or systems for reliably inserting foreign DNA into host cells. Such methods can result in transient expression of non-integrated transferred DNA, extra-chromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells. (See, e.g., U.S. Pat. No. 5,399,346.)

The term “vector” as used herein refers to any genetic element, such as a plasmid, phage, transposon, cosmid, chromosome, virus, virion, and the like, which is capable of replication when associated with the proper control elements and which can transfer gene sequences into cells. Thus, the term includes cloning and expression vehicles, as well as viral vectors.

The term “AAV vector” as used herein refers to a vector derived from an adeno-associated virus serotype, including but not limited to, AAV-1, AAV-2, AAV-3, AAV-4, AAV-5, AAVX7, and the like. AAV vectors can have one or more of the AAV wild-type genes deleted in whole or part, preferably the rep and/or cap genes, but retain functional flanking Inverted Terminal Repeat (ITR) sequences. Functional ITR sequences permit the rescue, replication and packaging of the AAV virion. Thus, an AAV vector is defined herein to include at least those sequences required for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The term “recombinant AAV virion,” or “rAAV virion” as used herein refers to an infectious, replication-defective virus composed of an AAV protein shell, encapsidating a nucleic acid of interest which is flanked on both sides by AAV ITRs. A rAAV virion is produced in a suitable host cell which has had an AAV vector, AAV helper functions and/or accessory functions introduced therein. In this manner, the host cell is rendered capable of encoding AAV proteins that are required for packaging the AAV vector (containing a recombinant nucleotide sequence of interest) into recombinant virion particles for subsequent gene delivery.

The term “transfection” is used herein refers to the uptake of an exogenous nucleic acid molecule by a cell. A cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid molecules into suitable host cells. The term refers to both stable and transient uptake of the nucleic acid molecule.

The term “transduction” or “transducing” as used herein refers to the delivery of a nucleic acid molecule to a recipient cell either in vivo or in vitro, via a replication-defective viral vector, such as a recombinant AAV virion.

The term “exogenous” as used herein refers to nucleic acid molecule such as gene sequences and control sequences, that are not normally not normally associated with a particular organism. Thus, a “exogenous” region of a nucleic acid molecule or a vector is a segment of nucleic acid within or attached to another nucleic acid molecule that is not found in association with the other molecule in nature. For example, a exogenous region of a nucleic acid construct could include a coding sequence flanked by sequences not found in association with the coding sequence in nature. Another example of a exogenous coding sequence is a construct where the coding sequence itself is not found in nature (e.g., synthetic sequences having codons different from the native gene). Similarly, a cell transformed with a construct which is not normally present in the cell would be considered exogenous for purposes of this invention.

The term “coding sequence” or a sequence which “encodes” or sequence “encoding” a particular protein, as used herein refers to a nucleic acid molecule which is transcribed (in the case of DNA) and translated (in the case of messenger mRNA) into a polypeptide in vitro or in vivo when placed under the control of appropriate regulatory sequences. A gene can include, but is not limited to, cDNA from prokaryotic or eukaryotic mRNA, genomic DNA sequences from prokaryotic or eukaryotic DNA, and even synthetic DNA sequences.

The term “regulatory sequence” is art-recognized and intended to include control elements such as promoters, enhancers and other expression control elements (e.g., polyadenylation signals), transcription termination sequences, upstream regulatory domains, origins of replication, internal ribosome entry sites (“IRES”), enhancers, enhancer sequences, post-regulatory sequences and the like, which collectively provide for the replication, transcription and translation of a coding sequence in a recipient cell. Not all of these regulatory sequences need always be present so long as the selected coding sequence is capable of being replicated, transcribed and translated in an appropriate host cell. Such regulatory sequences are known to those skilled in the art and are described in Goeddel, Gene Expression Technology: Methods in Enzymology 185, Academic Press, San Diego, Calif. (1990). It should be understood that the design of the viral vector may depend on such factors as the choice of the host cell to be transfected and/or the amount of protein to be expressed.

The term “promoter” is used herein refers to the art recognized use of the term of a nucleotide region comprising a regulatory sequence, wherein the regulatory sequence is derived from a gene which is capable of binding RNA polymerase and initiating transcription of a downstream (3′-direction) coding sequence.

The term “operably linked” as used herein refers to an arrangement of elements wherein the components are configured so as to perform their usual function. Thus, control elements operably linked to a coding sequence are capable of effecting the expression of the coding sequence. The control elements need not be contiguous with the coding sequence, so long as they function to direct the expression of the coding sequence. For example, intervening untranslated yet transcribed can be present between a promoter sequence and the coding sequence and the promoter sequence can still be considered “operably linked” to the coding sequence.

The terms “5′”, “3′”, “upstream” or “downstream” are art recognized terms that describe the relative position of nucleotide sequences in a particular nucleic acid molecule relative to another sequence.

The term “isolated” as used herein, refers to a nucleic acid or an amino acid sequence which is substantially free of other nucleic acid molecules or amino acid sequences, respectively.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The invention is described in more detail in the following subsections:

I Nucleic Acids Encoding Excitatory Amino Acid Transporter Proteins

The nucleotide sequences of excitatory amino acid transporter (EAAT) proteins, EAAT-1, EAAT-2, EAAT-3, EAAT-4, and EAAT-5, are known. The entire sequences for EAAT-1 is described by Kawakami et al. (1994) Biochem. Biophys. Res. Comm. 199: 171-176 (GenBank Accession No: NM 004172); for EAAT2 by Arriza et al., (1994) J. Neurosci. 14:5559-5569 (GenBank Accession No: NM 004171); for EAAT-3 by Ariza et al., ((1994) J. Neurosci. 14:5559-5569 (GenBank Accession No: NM 004170); for EAAT-4 by Fairman et al., (1995) Nature 375:599-603 (GenBank Accession No: NM U18244); and EAAT-5 by Arriza et al., (1997) Proc. Natl. Acad. Sci. 94:4155-4160 (GenBank Accession No: U76362). In a preferred embodiment, the isolated nucleic acid molecule comprises the nucleotide sequence of EAAT2 (SEQ ID NO: 1) which encodes the amino acid sequence of the EAAT2 protein (SEQ ID NO: 2).

Other embodiments also within the scope of the invention include a nucleic acid molecule encoding a fragment of the EAAT2 protein. In one embodiment, the fragment comprises an amino acid sequence from amino acid 73 to amino acid 210 of SEQ ID NO: 2. In another embodiment, the fragment comprises an amino acid sequence from amino acid 332 to amino acid 501 of SEQ ID NO: 2. In another embodiment, the fragment comprises an amino acid sequence from amino acid 388 to amino acid 414 of SEQ ID NO: 2. In another embodiment, the fragment comprises an amino acid sequence from amino acid 427 to amino acid 447 of SEQ ID NO: 2.

Also within the scope of the invention is an EAAT2 protein, or a fragment thereof that is homologous to SEQ ID NO: 2. To determine the homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J Mol. Biol. (48):444-453) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In another example, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In yet another example, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The skilled artisan will appreciate that changes can be introduced by mutation into the nucleotide sequences of SEQ ID NO: 1, thereby leading to changes in the amino acid sequence of the encoded EAAT2 protein SEQ ID NO:2, without altering the functional ability of the EAAT2 protein. For example, the nucleotide substitutions leading to amino acid substitutions at “non-essential” amino acid residues can be made in the sequence of SEQ ID NO: 1. A “non-essential” amino acid residue is a residue that can be altered from the wild-type sequence of EAAT2 (i.e., the sequence of SEQ ID NO:2) without altering the biological activity, whereas, an “essential” amino acid residue is required for biological activity. Accordingly, the invention also includes nucleic acid molecules encoding the EAAT2 protein that contains changes in the amino acid residues that are not essential for activity. Such proteins differ in amino acid sequence from SEQ ID NO:2, yet retain biological activity. In one embodiment, the isolated nucleic acid molecule comprises a nucleotide sequence encoding a protein wherein the protein comprises an amino acid sequence at least about 60%, 65%, 70%, 75%, 80%, 85%, 90%, 95%, 98% homologous to SEQ ID NO:2, or a fragment thereof.

Mutations or alterations to the nucleotide sequence can be introduced using standard molecular biology techniques. (See e.g., Sambrook et al. Molecular Cloning: A Laboratory Manual, 2nd Edition, Cold Spring Harbor Laboratory press (1989)). For example, mutations can be introduced by site directed mutagenesis, and PCR mutagenesis, additions and deletions. Preferably, conservative amino acid substitutions are made at one or more predicted non-essential amino acid residues. A “conservative amino acid substitution” is one in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

II Viral Vectors

The invention features a method of using adeno-associated viral vectors for gene therapy. AAV vectors can be constructed using known techniques to provide at least the operatively linked components of control elements including a transcriptional initiation region, a exogenous nucleic acid molecule, and a transcriptional termination region. The control elements are selected to be functional in the targeted cell. The resulting construct which contains the operatively linked components is flanked at the 5′ and 3′ region with functional AAV ITR sequences.

The nucleotide sequences of AAV ITR regions are known. The ITR sequences for AAV-2 are described, for example by Kotin et al. (1994) Human Gene Therapy 5:793-801; Berns “Parvoviridae and their Replication” in Fundamental Virology, 2nd Edition, (B. N. Fields and D. M. Knipe, eds.) The skilled artisan will appreciate that AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including but not limited to, AAV-1, AAV-2, AAV-3, AAV-4, AAV-5, AAVX7, and the like. Furthermore, 5′ and 3′ ITRs which flank a selected nucleotide sequence in an AAV expression vector need not necessarily be identical or derived from the same AAV serotype or isolate, so long as the ITR's function as intended, i.e., to allow for excision and replication of the bounded nucleotide sequence of interest when AAV rep gene products are present in the cell.

The skilled artisan can appreciate that regulatory sequences can often be provided from commonly used promoters derived from viruses such as, polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used include, for example, the early cytomegalovirus promoter Boshart et al. (1985) Cell 41:521-530, herpesvirus thymidine kinase (HSV-TK) promoter (McKnight et al. (1984) Cell 37:253-262), β-actin promoters (e.g., the human β-actin promoter as described by Ng et al. (1985) Mol. Cell Biol. 5:2720-2732) and colony stimulating factor-1 (CSF-1) promoter (Ladner et al., (1987) EMBO J. 6:2693-2698).

Alternatively, the regulatory sequences of the AAV vector can direct expression of the EAAT2 protein preferentially in a particular cell type, i.e., tissue-specific regulatory elements can be used. Non-limiting examples of tissue-specific promoters which can be used include, central nervous system (CNS) specific promoters such as, neuron-specific promoters (e.g., the neurofilament promoter; Byrne and Ruddle (1989) Proc. Natl. Acad. Sci. USA 86:5473-5477) and glial specific promoters (Morii et al., (1991) Biochem. Biophys Res. Commun. 175:185-191). Preferably, the promoter is tissue specific and is essentially not active outside the central nervous system, or the activity of the promoter is higher in the central nervous system that in other systems. For example, a promoter specific for the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. The promoter may be specific for particular cell types, such as neurons or glial cells in the CNS. If it is active in glial cells, it may be specific for astrocytes, oligodentrocytes, ependymal cells, Schwann cells, or microglia. If it is active in neurons, it may be specific for particular types of neurons, e.g., motor neurons, sensory neurons, or interneurons.

Suitable neuronal specific promoters include, but are not limited to, neuron specific enolase (NSE) (Olivia et al., (1991) Genomics 10:157-165, GenBank Accession No: X51956), human neurofilament light chain promoter (NEFL) (Rogaev et al., (1992) Hum. Mol. Genet. 1:781, GenBank Accession No: L04147), synapsis (Sauerwald et al., (1990) J. Biol. Chem. 265:14932-14937, GenBank Accession No: M55301), and serotonin receptor (Bloem et al., (1993) Brain Res. 17:194-200, GenBank Accession No: S62283). Glial specific promoters include, but are not limited to, glial fibrillary acidic protein (GFAP) promoter (Morii et al., (1991) Biochem. Biophys Res. Commun. 175:185-191, GenBank Accession No:M65210), S100 promoter (Morii et al., (1991) Biochem. Biophys Res. Commun. 175:185-191, GenBank Accession No: M65210) and glutamine synthase promoter (Van den et al., (1991) Biochem. Biophys. Acta. 2:249-251, GenBank Accession No:X59834). In a preferred embodiment, the nucleotide sequence encoding the EAAT2 protein or a portion thereof, is flanked upstream (i.e., 5′) by the neuron specific enolase (NSE) promoter. In another preferred embodiment, the nucleotide sequence encoding the EAAT2 protein or a portion thereof, is flanked upstream (i.e., 5′) by the glial fibrillary acidic protein (GFAP) promoter. In another preferred embodiment, the nucleotide sequence encoding the EAAT2 protein or a portion thereof, is flanked upstream (i.e., 5′) by the neuron specific enolase (NSE) promoter.

The AAV vector harboring the exogenous nucleic acid molecule flanked by AAV ITRs, can be constructed by directly inserting the exogenous nucleic acid molecule into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1:165-169; and Zhou et al. (1994) J. Exp. Med. 179:1867-1875).

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., Supra. Several AAV vectors are available from the American Type Culture Collection (“ATCC”) under Accession Numbers 53222, 53223, 53224, 53225 and 53226.

In order to produce rAAV virions, an AAV vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, a laboratory manual, Cold Spring Harbor Laboratories, N.Y., Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

Suitable host cells for producing rAAV virions include, but are not limited to, microorganisms, yeast cells, insect cells, and mammalian cells, that can be, or have been, used as recipients of a exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed. Non-limiting examples include CHO dhfr⁻ cells (Urlaub and Chasin (1980) Proc. Natl. Acad. Sci. USA 77:4216-4220), 293 cells (Graham et al. (1977) J. Gen. Virol. 36:59) or myeloma cells like SP2 or NS0 (Galfre and Milstein (1981) Meth. Enzymol. 73(B):3-46). The host cell can also be a mammalian neuronal cell line that includes glial cells, astrocytes and spinal cord neurons. In addition to cell lines, the invention is applicable to normal cells, such as cells to be modified for gene therapy purposes or embryonic cells modified to create a transgenic or homologous recombinant animal. Examples of cell types of particular interest for gene therapy purposes include hematopoietic stem cells, myeloid stem cells, glial cells, cord blood cells, fetal stem cells, pig stem cells, and neuronal cells.

In one embodiment, cells from the stable human cell line, 293 (readily available through, e.g., the ATCC under Accession No. ATCC CRL1573) are preferred in the practice of the present invention. Particularly, the human cell line 293, which is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Host cells containing the above-described AAV vectors are preferably rendered capable of providing AAV helper functions in order to replicate and encapsidate the exogenous nucleic acid molecule flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one, or both of the major AAV open reading frames (ORFs), namely the rep and cap coding regions, or functional homologues thereof.

The term “AAV rep coding region” as used herein refers to the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other exogenous) promoters. The Rep expression products are collectively required for replicating the AAV genome. For a description of the AAV rep coding region, see, e.g., Muzyczka (1992) Current Topics in Microbiol. and Immunol. 158:97-129; and Kotin (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

The term “AAV cap coding region” as used herein refers to the art-recognized region of the AAV genome which encodes the capsid proteins VP1, VP2, and VP3, or functional homologues thereof. These cap expression products supply the packaging functions which are collectively required for packaging the viral genome. For a description of the AAV cap coding region, See, e.g., Muzyczka (Supra).

AAV helper functions can be introduced into the host cell by transfecting the host cell with an AAV helper construct either prior to, or concurrently with, the transfection of the AAV vector, AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. (See, e.g., Samulski et al. (1989) J. Virol. 63:3822-3828; and McCarty et al. (1991) J. Virol. 65:2936-2945). A number of other vectors have been described which encode Rep and/or Cap expression products. See, e.g., U.S. Pat. No. 5,139,941.

Both AAV vectors and AAV helper constructs can be constructed to contain one or more optional selectable markers. Suitable markers include genes which confer antibiotic resistance or sensitivity to, impart color to, or change the antigenic characteristics of those cells which have been transfected with a nucleic acid construct containing the selectable marker when the cells are grown in an appropriate selective medium. Several selectable marker genes that are useful in the practice of the invention include the gene encoding Aminoglycoside phosphotranferase (APH) that allows selection in mammalian cells by conferring resistance to G418 (available from Sigma, St. Louis, Mo.). Other suitable markers are known to those of skill in the art.

As a consequence of the infection of the host cell with a helper virus, the AAV Rep and/or Cap proteins are produced. The Rep proteins also serve to duplicate the AAV genome. The expressed Cap proteins assemble into capsids, and the recombinant AAV genome is packaged into the capsids. This results in AAV replication, and the DNA is packaged into rAAV virions. Following recombinant AAV replication, rAAV virions can be purified from the host cell using a variety of conventional purification methods, such as CsC1 gradients. The resulting rAAV virions are then ready for use for DNA delivery to various cell types.

Alternatively, a vector of the invention can be a virus other than the adeno-associated virus, or portion thereof, which allows for expression of a nucleic acid molecule introduced into the viral nucleic acid. For example, replication defective retroviruses, adenoviruses and lentivirus can be used. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. The genome of adenovirus can be manipulated such that it encodes and expresses the protein of interest but is inactivated in terms of its ability to replicate in a normal lytic viral life cycle. See e.g., Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art.

III Delivery Systems

Delivery systems include methods of in vitro, in vivo and ex vivo delivery of the rAAV virions or AAV vectors. Generally, rAAV virions or AAV vectors can be introduced into regions of the CNS using in vivo transduction techniques. Particularly, for in vivo delivery, the rAAV virions will be formulated into pharmaceutical compositions and generally administered by injection directly into the central nervous system (CNS).

For in vivo delivery of vectors of the invention to cells, the vector can be administered to a subject in a pharmaceutically acceptable carrier. The term “pharmaceutically acceptable carrier”, as used herein, refers to any physiologically acceptable carrier for in vivo administration of the vectors of the present invention.

Such carriers do not induce an immune response harmful to the individual receiving the composition. Pharmaceutically acceptable carriers include, but are not limited to, liquids such as water, saline, aqueous buffer solutions, glycerol, ethanol, solvents, dispersion media, antibacterial and antifungal agents, isotonic and absorption delaying agents, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A review of suitable pharmaceutically acceptable excipients is available in Remington's Pharmaceutical Sciences (Mack Pub. Co., N.J. 1991). The use of such media and agents is well known in the art. In all cases, the pharmaceutical composition must be sterile and must be fluid to the extent that easy syringability exists. It must be stable under the conditions of manufacture and storage and must be preserved against the contaminating action or microorganisms such as bacteria and fungi. Prevention of the action of microorganisms can be achieved by various anti-bacterial and anti-fungal agents, for example, parabens, chlorobutanol, phenol, ascorbic acid, thimerosal, and the like.

Appropriate doses will depend, among other factors, on the mammal being treated (e.g., human or nonhuman primate or other mammal), age and general condition of the subject to be treated. An appropriate effective amount can be readily determined by one of skill in the art. The skilled artisan will appreciate that a “therapeutically effective amount” will fall in a relatively broad range that can be determined through clinical trials. For example, a therapeutically effective dose may be on the order of from about 10³ to 10¹⁵ of the AAV vector. Other effective dosages can be readily established by one of ordinary skill in the art through routine trials establishing dose response curves. Dosage treatment may be a single dose schedule or a multiple dose schedule. Moreover, the subject may be administered as many doses as appropriate. One of skill in the art can readily determine an appropriate number of doses.

For a more widespread distribution of the AAV vector throughout a wide region of the CNS, it may desirable to inject vectors into the cerebrospinal fluid, e.g., by lumbar puncture (See e.g., Kapadia et al., (1996) Neurosurg 10:585-587). The vector may also be delivered intracerebroventricularly and/or intrathecally, for specific applications. The AAV vector can also be delivered to regions of the central nervous system, such as the cells of the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof.

In a preferred embodiment, the AAV vector can also be delivered to the target cells (e.g., astrocytes) of the cervical spinal cord ventral horn by percutaneous spinal cord injection. This techniques involves the direct injection of the vector to the spinal cord. The appropriate dosages and volume of vector selected for injection can be determined empirically, as is routinely practiced in the art. For example, the volume can be determined based on the cross sectional area of the spinal cord. The human spinal cord cross sectional area is approximately 8 mm (AP)×15 mm (right to left). As spinal cords are essentially cylinders, their volume per unit length is proportional to their cross sectional area. The volume per unit for a human cervical cord is approximately 120 mm²L. A suitable volume for a vector injection can be 70 microliters. The speed at which injection is delivered is important both to the safety and efficacy of delivery. The injections can be delivered as a bolus injection, or as a timed delivery injection. A suitable speed for a timed injection utilizing a micropump can be about 10 minutes.

Alternatively, precise delivery of the AAV vector into specific sites of the brain, e.g., into regions comprising glial cells or other intracranial regions, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for AAV vector microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. A pharmaceutical composition containing an AAV vector can then be microinjected at the selected target sites. Spread of the AAV vector from the site of injection will be a function of passive diffusion which may be controlled by adjusting the ratio of the recombinant virion in the pharmaceutical composition.

For targeting the AAV vector to a particular type of cell, e.g., a neuron, it may be necessary to associate the vector to a targeting agent that binds specifically to a surface receptor of the cell. For example, the vector may be conjugated to a ligand (e.g., enkephalin) for which certain nervous system cells have receptors. The conjugation can be covalent, e.g., by using a cross linking agent such as glutaraldehyde, or non-covalent, e.g., by binding an avidinated ligand to a biotinylated vector. Other methods of targeting to the desired target site include using the nuclear localization signal (NLS) peptide (See e.g., Zanta et al., (1999) Proc. Natl. Acad. Sci. 96:91-96 and Aronsohn et al., (1998) J. Drug Target 5:163-169).

Recombinant virions or AAV vectors can also be transferred into cells in vivo, for example by application of a delivery mechanism suitable for introduction of nucleic acid into cells in vivo, such as receptor-mediated DNA uptake (see e.g., Wu and Wu, (1988) J. Biol. Chem. 263:14621; Wilson et al. (1992) J. Biol. Chem. 267:963-967; and U.S. Pat. No. 5,166,320), direct injection of DNA (see e.g., Accede et al. (1991) Nature 332:815-818; and Wolff et al. (1990) Science 247:1465-1468) or particle bombardment (see e.g., Cheng et al. (1993) Proc. Natl. Acad. Sci. USA 90:4455-4459; and Zelenin, et al. (1993) FEBS Letters 315:29-32).

Alternatively, the nucleic acid molecule can be delivered using a non-viral delivery system. This includes delivery of the AAV vector to the desired tissues in colloidal dispersion systems that include, for example, macromolecule complexes, nanocapsules, microspheres, beads, and lipid-based systems including oil-in-water emulsions, micelles, mixed micelles, and liposomes. The preferred colloidal system of this invention is a liposome.

Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (See e.g., Fraley, et al., (1981) Trends Biochem. Sci., 6:77). In addition to mammalian cells, liposomes have been used for delivery of nucleic acid molecules in plant, yeast and bacterial cells. In order for a liposome to be an efficient gene transfer vehicle, the following characteristics should be present: (1) encapsulation of the genes encoding the nucleic acid molecule at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., (1988) Biotechniques, 6:682).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. Additional examples of lipids include, but are not limited to, polylysine, protamine, sulfate and 3b -[N-(N′,N′dimethylaminoethane) carbamoyl] cholesterol.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Another alternative is the direct delivery of naked DNA encoding the EAAT2 protein. The naked DNA can be administered using an injection gun (See e.g., Accede et al. (1991) Nature 332:815-818; and Wolff et al. (1990) Science 247:1465-1468 Murashatsu et al., (1998) Int. J. Mol. Med. 1:55-62; Agracetus et al., (1996) J. Biotechnol. 26: 37-42; Johnson et al., (1993) Genet. Eng. 15:225-236).

IV Gene Therapy

The invention is particularly useful for gene therapy purposes, in treatments for either genetic or acquired diseases. The general approach of gene therapy involves the introduction of nucleic acid into cells such that one or more gene products encoded by the introduced genetic material are produced in the cells to restore or enhance a functional activity. For reviews on gene therapy approaches see e.g., Anderson (1992) Science 256:808-813; Miller (1992) Nature 357:455-460; Friedmann (1989) Science 244:1275-1281; Cournoyer, et al. (1990) Curr. Opin. Biotech. 1:196-208; During et al., (1998) Molec. Med. Today 4:485-493; Cornetta et al., (1989) Prog. Nucleic Acid Res. Mol. Biol. 36:311-322; Anderson (1995) Sci. Amer. 273:124-128).

To use the system of the invention for gene therapy purposes, in one embodiment, cells of a subject in need of gene therapy are modified to contain a nucleic acid molecule encoding an EAAT2 protein. The cells of the subject can be modified ex vivo and then introduced into the subject or the cells can be directly modified in vivo. Expression of the EAAT2 gene in the cells of the subject is preferably constitutive.

Genes of particular interest to be expressed in cells of a subject for treatment of genetic or acquired diseases include those encoding EAAT-1, EAAT-2, EAAT-3, EAAT-4, EAAT-5, and glutamine synthase. Most preferably, the gene encoding EAAT2. Cells types which can be modified for gene therapy purposes include hematopoietic stem cells, myeloid stem cells and glial cells. For further descriptions of cell types, genes and methods for gene therapy see e.g., Wilson, et al. (1988) Proc. Natl. Acad. Sci. USA 85:3014-3018; Armentano. et al. (1990) Proc. Natl. Acad. Sci. USA 87:6141-6145; Wolff et al. (1990) Science 247:1465-1468; Chowdhury et al. (1991) Science 254:1802-1805; Ferry et al. (1991) Proc. Natl. Acad. Sci. USA 88:8377-8381; Wilson et al. (1992) J. Biol. Chem. 267:963-967; Quantin et al. (1992) Proc. Natl. Acad. Sci. USA 89:2581-2584; Dai et al. (1992) Proc. Natl. Acad. Sci. USA 89:10892-10895; van Beusechem et al. (1992) Proc. Natl. Acad. Sci. USA 89:7640-7644; Rosenfeld et al. (1992) Cell 68:143-155; Kay et al. (1992) Human Gene Therapy 3:641-647; Cristiano et al. (1993) Proc. Natl. Acad. Sci. USA 90:2122-2126; Hwu et al. (1993) J. Immunol. 150:4104-4115; and Herz and Gerard (1993) Proc. Natl. Acad. Sci. USA 90:2812-2816).

Gene therapy applications of particular interest in ALS include over expression of the EAAT2 gene in glial cells to increase to level of the EAAT2 protein in such cells. This increased expression of the EAAT2 in glial cells can convert toxic levels of glutamate into a non-toxic levels and thereby suppress the effects of glutamate toxicity. Gene therapy applications of the invention, allow for the stable long term expression of the EAAT2 gene and avoids the need for continued administration of a gene product of interest at intermittent intervals. This approach reduces the need for repeated injections of a gene product, which may be painful and/or cause side effects and would likely require continuous visits to a physician.

Animal models used in the study of cerebral ischemia, epilepsy, and trauma have demonstrated that neuronal cell death is caused by excessive neurotransmission. Accordingly, the gene therapy applications of the present invention may also benefit disorders that include, but are not limited to, epilepsy, stroke, cerebral ischemia and trauma.

Epilepsy

Epileptic seizures are the outward manifestation of excessive and/or hypersynchronous abnormal activity of neurons in the cerebral cortex. The behavioral features of a seizure reflects the function of a portion of the cortex where the hyper activity is occurring. There are different types of seizures, for example, generalized seizures involve the entire brain simultaneously. Generalized seizures can result in the loss of conscious awareness (referred to as “petit mal” seizure), or may result in a convulsion with tonic-clonic contractions of the muscles (referred to as “grand mal” seizure). There are also partial seizures which begin in one part of the brain and remain localized. The subject typically remains conscious throughout the seizure. If the subject loses consciousness, the seizure is referred to as a “complex partial seizure”.

A number of animal models indicate the involvement of the substantia nigra, a particular portion of the brain considered to be part of neural circuitry referred to as the basal ganglia (See e.g., Gale (1985) Fed. Proc. 44:2414-2424 and Depaulis, Vergnes et al. (1994) Prog. Neurobiol. 42:33-52). Inhibition of the substantia nigra will increase the threshold for seizure occurrence in experimental animal models of epilepsy (See e.g., Loscher et al. (1998) J. Neurosci Res. 51:196-209 and Tanaka et al. (1996) Brain Res. 737:59-63). The substantia nigra receives input from the subthalamic nucleus (STN) which is excitatory and involves glutamate as the neurotransmitter conveying information at the synapse (See e.g., Kitano et al. (1998) Brain Res. 784:228-38; and Alexander et al. Prog. Brain Res. 85:119-146). A lesion of the subthalamic nucleus will reduce the inhibitory output of the internal segment of the globus pallidus and substantia nigra reticulata (SN) (See Bergman et al., (1990) Science 249:1436-1438).

In another preferred embodiment, the method of the invention can be used to treat epilepsy. Both rat and monkey models of epilepsy are available in which effective therapies are predictive of therapeutic efficacy in humans. For example, rats which exhibit audiogenic seizures are commercially available. Once the epileptic focus of these rats is located, a nucleic acid encoding EAAT2 can be introduced at the epileptic focus. The ameliorative effects of increased EAAT2 expression can be monitored by EEG, or by monitoring the decrease in seizure occurrence.

Huntington 's disease

In another preferred embodiment, the method of the invention can be used to treat a subject with Huntington's disease. Models of Huntington's disease have been developed in several different animals. For example, rat (Isacson et al. (1985) Neuroscience 16:799-817), monkey (Kanazawa, et al. (1986) Neurosci. Lett. 71:241-246), and baboon (Hantraye. et al. (1992) Proc. Natl. Acad. Sci. USA 89:4187-4191; Hantraye, et al. (1990) Exp. Neurol. 108:91-014; Isacson, et al. (1989) Exp. Brain Res. 75(1):213-220) models of Huntington's disease have been described in which effective therapies are predictive of therapeutic efficacy in humans. Neurodegeneration in Huntington's disease typically involves degeneration in one or both nuclei forming the stratium or corpus stratium, the caudate nucleus and putamen. Increased levels of EAAT2 in specific affected brain regions can be used to provide neuroprotection.

Morphological and immunohistochemical studies can then be performed by conventional techniques to determine whether the expressed EAAT2 provided neuroprotection. Behavioral tests can also be performed using standard techniques, such as the Barnes Circular Maze, the Circular track mobility test, and the Contextual Fear Conditioning test (See e.g., Barnes et al. (1979) J. Comp. Physiol. Psychol. 93:74-104; Ellis et al., (1992) Exp. Neurol. 115:376-87; 19: Hantraye et al., (1990) Exp. Neurol. 108:91-104; Rudy et al., (1999) Behav. Neurosci. 113:867-80 and Winson (1978) Science, 201:160-163).

Parkinson's disease

Parkinson's disease in humans primarily affects subcortical structures, especially the substantia nigra and loercus caeruleus. Several animal models of Parkinson's disease have been generated in which effective therapies are indicative of therapeutic efficacy in humans. These animal models include three rat models (the rats having lesions in substantia nigral dopaminergic cells caused by treatment with 6-hydroxydopamine, 1-methyl-4-phenyl-1,2,3,6-tetrahydropyridine (MPTP), or surgical transection of the nigral striatal pathway) (See, e.g. Björklund et al. (1982) Nature 298:652-654), a rhesus monkey model (the monkeys having lesions in substantia nigral dopaminergic cells caused by treatment with MPTP) (See, e.g., Smith, et al. (1993) Neuroscience 52):7-16; Bakay et al. (1985) Appl. Neurophysiol. 48:358-361; Zamir. et al. (1984) Brain Res. 322:356-360), and a sheep model (the sheep having lesions in substantia nigral dopaminergic cells caused by treatment with MPTP) (Baskin, et al. (1994) Life Sci. 54:471-479). In another embodiment, the method of the invention can be used to treat a subject with Parkinson's disease. To assess therapeutic strategies, morphological and immunohistochemical studies can be performed by conventional techniques. Behavioral tests can also be performed as described above.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Preparation of a Gene Transfer Vector

The vector is an adeno-associated vector expressing the human 1.8 kb EAAT2 cDNA under the control of the 2.2 kb glial fibrillary acidic protein (GFAP) promoter. The EAAT2 gene and the promoter are flanked by the AAV 145 bp inverted terminal repeats (ITR). The vector can be constructed using standard recombinant DNA techniques (See Sambrook et al. Molecular Cloning: A Laboratory Manual, 2nd Edition, Cold Spring Harbor Laboratory press (1989)).

### Example 2

### Packaging Protocol for the Production of Recombinant Virions

Recombinant AAV-GFAP-EAAT2 virions (rAAV-GFAP-EAAT2) can be generated using the helper-free packaging system as previously described by During, M. J. et al. (1998) Nature Med. 4:1131-113, with a slight modification. Human embryonic 293 kidney cells (from a Master Cell Bank), passages #4-12 only, were used for packaging. 1.5×10⁷ cells were seeded into 40×15 cm dishes overnight in complete DMEM (Gibco) containing 10% fetal bovine serum (Hyclone), 0.1 mM MEM non-essential amino acids solution (Gibco), 1 mM MEM sodium pyruvate (Gibco), 0.05% Penicillin-Streptomycin (5,000 units/ml, Gibco). When close to 70% confluent (2-3 hours prior to transfection), the cells were fed with fresh Iscove modified DMEM (Gibco) containing 10% fetal bovine serum without antibiotics. All plasmids were prepared by alkaline lysis, further purified by HPLC (BioCAD, Sprint, PerSeptive Biosystems), and concentrated by 2 volumes of 100% ethanol (AR grade, BDH). A11 HPLC elute buffers (Buffer A: 250 mM TrisHCl, 10 mM EDTA, pH 8.0; Buffer B: 25 mM TrisHC1, 1 mM EDTA, 2M NaCl, pH, 8.0; Buffer C: Milli Q water) used for purification was autoclaved and filtered prior to usage. For each 15 cm tissue culture plate, a total of 60 μg of plasmid DNA is dissolved in 3.0 ml of 0.25M CaCl₂ and then quickly mixed with 3.0 ml of HEPES-buffered saline (50 mM HEPES, 280 mM NaCl, 1.5 mM Na₂HPO₄ [pH 7.10]), incubated for 2 min and added to the cells. At 6-8 hours after transfection, the medium was aspirated and cells were washed with IMDM supplemented with 10% fetal bovine serum without antibiotics; the washing medium was aspirated and replaced with fresh IMDM (Gibco) containing 10% fetal bovine serum with trace antibiotics. The cells are harvested 48 hours after transfection. After low-speed centrifugation on a tabletop centrifuge, the cell pellets were resuspended in 150 mM NaCl, 20 mM Tris pH 8.0. Cell debris was removed with low speed centrifugation. The clarified supernatant was collected into a 25 ml polypropylene tube containing 0.5% sodium deoxycholate and Benzonase 5 U/ml. The cells were then incubated in a 37° C. waterbath for 30-40 min, before removing cell debris by centrifugation at 3000 g×15 min, 4° C. Supernatants were heated at 56° C. for 15 min, then frozen in a dry ice/ethanol bath, and thawed before removal of cell debris by centrifugation at 3000 g×15 min, 4° C. Supernatants were filtered through a 32 mm 0.45 um Acrodisc syringe filter to remove any particulate matter, prior to heparin column purification. Supernatants were run at a flow rate of 2 ml/min through a heparin column (1 ml HiTrap heparin columns, Sigma #5-4836). Columns were washed with 20 ml 100 mM NaCl, 20 mM Tris (pH 8.0). The elution fractions are spun down to as small a volume as possible. The fractions were dialyzed overnight against 1XPBS with 1 mM MgCl₂, using a dialysis membrane (Pierce Slide-A-Lyzer dialysis cassettes, 3500 MWCO). Purified virus on a protein gel was stained with silver stain to determine the purity.

### Example 3

### Preparation of Primary Neuronal Cultures

In order to obtain neuronal cultures, primary cortical, striatal, hippocampal and nigral (mesencephalic) cell cultures can be prepared from E15 pregnant Wistar rats. The embryos from two litters (approximately 24 embryos in total) can be removed into a dish of warm dissecting medium (Ca²⁺⁻ and Mg²⁺⁻ free Hank's balanced salt solution containing 0.6% glucose, 100 U/ml penicillin, 100 μg/ml streptomycin, 15 mM HEPES). The appropriate brain regions can be dissected out and collected into tubes containing fresh dissecting medium. Tissue can then be digested in a trypsin solution (0.25% trypsin, 200 μg/ml DNase in dissecting medium) for 15 minutes at 37° C. in a shaking waterbath. The trypsin reaction can be terminated by the addition of trypsin inhibition medium (100 μg/ml soybean trypsin inhibitor, 20% fetal bovine serum, 200 μg/ml DNase in dissecting medium), and the tissue can be washed twice in wash medium (dissecting medium containing 10% fetal bovine serum and 200 μg/ml DNase). A cell suspension can then be obtained by triturating the tissue using a small-bore, fire-polished Pasteur pipette until tissue clumps are no longer visible, and then the suspension can be filtered through a 100 μm nylon filter to eliminate any remaining cell clumps. The cells can then be pelleted at 400 g, resuspended, counted and plated onto poly-1-lysine-coated dishes at a density of 250,000 cells/well in 24 well-plates and at a density of 100,000 cells/well in 96 well-plates in Neurobasal™ medium (Gibco BRL) containing B27 supplement (Gibco BRL) and 0.5 mM 1-glutamine. Medium can be replenished every 48 hours.

### Example 4

### Vector Characterization

(a) Determination of rAAV Titer with Optical Density (O.D.) Reading

Estimation of viron concentration was accomplished by UV spectrophotometric analysis of extracted viral genomic DNA, using the formula that one absorbance unit at 260 nm is equal to 10¹² viral particles per ml (Curiel et al., 1991), where 1 mg viral protein is equal to 3.4-3.5×10¹² viral particles.

(b) Determination of rAAV Titer with ELISA

A sandwich ELISA technique was used to determine rAAV titres. A monoclonal antibody specific for AAV assembled capsids was coated onto microtiter strips and was used to capture AAV particles. A biotin-conjugated monoclonal antibody to AAV was bound to the immune complex, and streptavidin peroxidase conjugate reacted with the biotin molecules. Addition of substrate solution resulted in a color reaction which was proportional to specifically bound virus particles, and allowed the quantitative determination of an unknown particle titer (Wistuba et al., 1997). The AAV titration ELISA kit was provided by Progen (Germany). One 100 μl of ready-to-use wash buffer, positive and negative controls and dilutions of standard and samples were pipetted into appropriate wells of the microtiter strips, and strips were sealed with adhesion foil. After incubation for 1 hour at 37° C., the solution was removed and each well was rinsed 3 times with 200 μl of washing buffer for 30 seconds. 100 μl of ready-to-use biotin conjugate was added, and strips were sealed with adhesion foil and incubated for one hour at 37° C., washing steps were repeated as above. 100 μl of ready-to-use streptavidin conjugate was added, and strips were sealed with adhesion foil and incubated for one hour at 37° C. Washing steps were repeated as described above. 100 μl of substrate was pipetted into each well and incubated at room temperature for 10 min. The reaction was stopped by adding 100 μl of stop solution into each well. Absorbance is measured photometrically at 450 nm.

(c) Determination of AAV Particle to Transducing Unit Ratio

To test for the AAV transducing unit ratio, 5×10⁴ 293 cell per well were seeded onto collagen-coated 24 well plates. The cells were grown in DMEM (Gibco) containing 10% fetal bovine serum (Hyclone), 0.1 mM MEM non-essential amino acids solution, 1 mM MEM sodium pyruvate, 0.05% Penicillin-Streptomycin (5,000 units/ml), at 5% CO₂ and 37° C. overnight. 0.5 μl of AAV/GFAP EAAT2 virus was added and incubated for 48 hours. For rat primary neurons and glia, E15 animals were used for nigra and cortex preparation, while E18 animals were used for hippocampal and striatal primary cell preparation. 250,000 harvested cells per well were pipetted into poly-1-lysine-treated 24 well plates, and incubated at 5% CO₂ and 37° C. for 24-48 hours. Medium B (15% FCS, 0.6% glucose, 100U per ml pen/strep in DMEM/F12) was added on cultures. After 3 days incubation, 0.5 μl of AAV-NSE-EAAT2 virus was added to cortical culture, and to nigral, hippocampual and striatal cultures after 4-5 days incubation. All media was replaced with fresh culture medium one day before addition of virus; cultures are incubated at 5% CO₂ and 37° C. for 3 days, then cells were fixed with 4% paraformaldehyde in 0.1 M phosphate buffer (pH 7.4) for 15 minutes and washed with phosphate buffered saline (PBS) containing TritonX100.

(d) Determination that rAAV is Stable to Dialysis

To determine rAAV virus stability during the dialysis processing, rAAV-CMV-lacZ virus was used for comparison. After ultracentrifugation, rAAV-CMV-lacZ from the same collection tube was divided and processed into two fractions, with and without dialysis, 1×10⁶ particles of rAAV virus per well, & added to 6-well plates seeded with 5×10⁵ 293 cell for 24 hours. After incubation at 37° C. for 2 days, enzyme activity is determined by β-Gal ELISA (Boehringer Mannheim). For rAAV-GFAP-EAAT2, 5×10⁴ human 293 cells were seeded onto a collagen-coated 24-well plate for 24 hours and 1×10⁵ virus particles with or without dialysis were added into each well & incubated for 3 days prior to fixation for immunocytochemistry. The recovery rate of rAAV-GFAP-EAAT2 virus during the dialysis is run in a separate experiment, and is estimated by adding trace of hemoglobin, ¹⁴C-methylated (Sigma) into samples for dialysis. A part of sample is taken to measure radioactivity by liquid scintillation counter (Wallac) before and after dialysis.

(e) Determination of 293 Cell Protein Level

Approximately 5-10×10⁸ particles were separated on a 10% SDS-PAGE gel and stained with Coomasie R250. This analysis shows the correct virion protein composition and presence of less than 1% contaminating cellular proteins. Protein bands were captured with a video camera and saved to a Macintosh computer file. Integrated densities from each band were measured and analyzed with the NIH Image software. Silver staining was also performed to analyze the protein content of vector stocks, which show ˜99% purity.

(f) Determination of Infectious Adenovirus

Lack of contamination with infectious adenovirus—2×10⁶ 293 cells (containing the E1A gene) were infected with 20 microliters (2×10¹⁰ particles) of the rAAV and incubated for three days at 37° C. in 5% CO₂. No cytopathic effect was obtained. An immunofluorescent assay was also employed, in which the infected cells were stained for adenovirus hexon protein in an immunofluorescence assay (Chemicon). The sensitivity of this test is 6 adenoviral particles in the presence of 6×10¹⁰ control rAAV, with no signal apparent in the vector stock.

(g) Analysis of Wild-Type AAV

The presence of wild-type AAV was determined by a PCR assay (see Snyder et al., 1997). 5×10¹⁰ rAAV particles (200 μl) were treated with DNase I (100 units/ml for 30 min at 37° C.) to degrade any unencapsulated DNA, with proteinase K (0.5 mg/ml for 60 minutes at 37° C.) to liberate the rAAV genomes, which were phenol extracted ×2, ethanol precipitated, and dissolved in 30 μl of water. 3 μl volume (˜6×10⁹ rAAV particles) is subjected to PCR along with positive and negative controls. The PCR primers were “D1” (5′-ACTCCATCACTAGGGGTTCC-3′) in the AAV ITR; “D2” (5′-GGTAATGATTAACCCGCCATGCTACTTATC-3′) also in the ITR; “AAV2S2” (5′TCAGAATCTGGCGGCAACTCCC-3′) in the AAV rep gene; “Splice1” (5′-TCGTCAAAAAGGCGTATCAG-3′) in the AAV splice region; “CAP2” (5′-TCCCTTGTCGAGTCCGTTGA-3′) in the AAV cap gene; and “CAP1” (5′-CAGAAGGAAAACAGCAAACG-3′) in the AAV cap gene. PCR is carried out under standard conditions with Taq polymerase for 25 cycles using the following primer pairs: D1 and AAV2S2 to detect left end sequences in wild-type AAV; D2 and AAV2S2 to specifically detect left end sequences in wild-type AAV arising from recombination between the vector and helper plasmids; Splice1 and CAP2 to detect internal sequences; and CAP1 and D2 to detect right end sequences in both wild-type AAV virus and wild-type AAV arising from recombination between the vector and helper plasmids. The products are separated on a 2% agarose gel and stained with ethidium bromide, and no bands indicative of a wild-type AAV contamination are detected to a sensitivity of one wild-type in 10⁹ rAAV.

The presence of wild-type AAV was also determined by a modified replication center assay as described by Snyder et al. (1996). Human 293 cells were seeded in a 24-well plate. All wells (1×10 ⁵ cells) were then co-infected with adenovirus at an MOI of 20 and also 10, 5, 2.5 or 1 ul of the rAAV stock. As a positive control, Ad-infected cells were infected with 1000, 100, 10 or 1 wtAAV particles, which were spiked into a rAAV stock already determined to be free of wild-type AAV. Cells were incubated for 24 hours; the cell culture supernatants (containing any detached cells) were removed and diluted into 10 ml PBS. The cells were detached from the plate with trypsin and combined with their corresponding supernatants. The single-cell suspension was vacuum filtered onto nylon membranes wetted with PBS, and the membranes were incubated cell side up on Whatman filter paper saturated with 0.5N NaOH/1.5M NaCl for 5 min at room temperature, followed by Whatman paper saturated with 1M Tris-HCl (pH 7.0)/2× SSC for 5 min at room temperature. The paper was probed with rep and cap sequences and exposed to film. No wild-type AAV was detected in the rAAV stock, to a sensitivity of 1 in 10⁹ rAAV, indicating a complete absence of wtAAV contamination.

(h) Delivery and Distribution

The method of delivery is important in obtaining efficient transduction of target cells in the mammalian brain. A recent study Cunningham et al., (1999) shows that vector concentration is a significant parameter for enhancing transduction. Therefore the quality and concentration of vector stocks was a critical. With titers of >10¹³ transducing particles/ml, delivery of 3 microliters at a single site can transduce a region extending several millimeters. Furthermore, microperfusion at slow flow rates using high titer will optimize expression for a given expression cassette and vector dose.

### Example 5

### In vivo Expression of EAAT2

Preliminary results from a transgenic mouse produced by a cross between a mutant ALS mouse and a mutant mouse that significantly overexpresses EAAT2, shows a substantial increase the life span of the animal. Overexpression of EAAT2 engineered with an amplified GFAP promoter in a 10X transgenic mouse was phenotypically normal. The 10xEAAT2 mouse was crossed with the familial ALS mouse model (SOD G93A). The resulting double-heterozygote offspring of that cross has now survived 12 months and is still showing no symptoms of the disease. This offspring is now 6.5 months beyond the normal life expectancy of a SOD g93A transgenic. The single-heterozygote SOD offspring of the cross that did not contain the over expression of EAAT2 died at the normal life span for a G93A mouse. For comparison purposes, data on a mouse on Rilutek (a glutamate release inhibitor) extended the life of the G93A mouse only by 2-3 weeks, and the longest a genetic cross has only extended the life of a G93A mouse by 3 months.

What is claimed is:

1. A method for treating a subject with a disorder associated with aberrant production of an excitatory amino acid transporter (EAAT) protein comprising:
transducing mammalian cells with a therapeutically effective amount of a recombinant virus which has encapsidated therein a gene transfer vector comprising:
(i) a first viral inverted terminal repeat sequence;
(ii) a nucleotide sequence encoding an EAAT protein operably linked to a promoter functional in stem cells;
(iii) a second viral inverted terminal repeat, wherein at least one of the elements (i) and (iii) comprises an AAV packaging signal; and
expressing the EAAT protein at levels that ameliorate the disorder associated with aberrant concentrations of an EAAT protein.

2. The method of claim 1, wherein the step of transducing mammalian cells further comprises transducing mammalian cells with a recombinant virus selected from the group consisting of adenovirus, adeno-associated virus, retrovirus and lentivirus.

3. The method of claim 1, wherein the step of transducing mammalian cells further comprises transducing mammalian cells selected from the group consisting of hematopoietic stem cells, myeloid stem cells, glial cells, cord blood cells, fetal stem cells, pig stem cells, and neuronal cells.

4. The method of claim 1, wherein the step of transducing mammalian cells further comprises transducing the mammalian cells ex vivo.

5. The method of claim 1, wherein the step of transducing mammalian cells further comprises transducing mammalian cells in vivo.

6. The method of claim 1, wherein said disorder is selected from the group consisting of amyotrophic lateral sclerosis (ALS), Epilepsy, Huntington's Disease and Parkinson's Disease.

7. A method for treating a subject with a disorder associated with aberrant production of an excitatory amino acid transporter (EAAT) protein comprising:
transducing stem cells with a therapeutically effective amount of a recombinant adeno-associated virus (AAV) virion which has encapsidated therein a gene transfer vector comprising:
(i) a first AAV inverted terminal repeat sequence;
(ii) a nucleotide sequence encoding an EAAT protein operably linked to a promoter functional in stem cells;
(iii) a second AAV inverted terminal repeat, wherein at least one of the elements (i) and (iii) comprises an AAV packaging signal; and
expressing the EAAT protein at levels that ameliorate the disorder associated with aberrant concentrations of an EAAT protein.

8. The method of claim 7, wherein the step of transducing stem cells further comprises transducing myeloid stem cells.

9. The method of claim 7, wherein the step of transducing stem cells further comprises transducing the stem cells ex vivo.

10. The method of claim 7, wherein the step of transducing stem cells further comprises transducing stem cells in vivo.

11. The method of claim 7, wherein the step of transducing stem cells further comprises transducing stem cells with an AAV virion which has encapsidated therein a gene transfer vector comprising a nucleotide sequence encoding an EAAT protein selected from the group consisting of EAAT1, EAAT2, EAAT3, EAAT4, and EAAT5.

12. The method of claim 7, wherein said promoter is selected from the group consisting of early cytomegalovirus promoter (CMV), herpesvirus thymidine kinase (TK) promoter and CSF-1 promoter.

13. The method of claim 7, wherein said disorder is selected from the group consisting of amyotrophic lateral sclerosis (ALS), Epilepsy, Huntington's Disease and Parkinson's Disease.

14. A method for treating amyotrophic lateral sclerosis (ALS) comprising:
transducing stem cells with a therapeutically effective amount of a recombinant adeno-associated virus (AAV) virion which has encapsidated therein a gene transfer vector comprising:
(i) a first AAV inverted terminal repeat sequence;
(ii) a nucleotide sequence encoding EAAT2 operably linked to a promoter functional in stem cells,
(iii) a second AAV inverted terminal repeat, wherein at least one of the elements (i) and (iii) comprises an AAV packaging signal; and
expressing the EAAT2 protein at levels that ameliorate ALS.

15. The method of claim 14, wherein the step of transducing stem cells further comprises transducing myeloid stem cells.

16. The method of claim 14, wherein the step of transducing stem cells further comprises transducing the stem cells ex vivo.

17. The method of claim 14, wherein the step of transducing stem cells further comprises transducing stem cells in vivo.

18. The method of claim 14, wherein said promoter is selected from the group consisting of early cytomegalovirus promoter (CMV), herpesvirus thymidine kinase (TK) promoter and CSF-1 promoter.

19. A method for treating amyotrophic lateral sclerosis (ALS) comprising:
transducing glial cells with a therapeutically effective amount of a recombinant adeno-associated virus (AAV) virion which has encapsidated therein a gene transfer vector comprising:
(i) a first AAV inverted terminal repeat sequence;
(ii) a nucleotide sequence encoding EAAT2 operably linked to a promoter functional in glial cells,
(iii) a second AAV inverted terminal repeat, wherein at least one of the elements (i) and (iii) comprises an AAV packaging signal; and
expressing the EAAT2 protein at levels that ameliorate ALS.

20. The method of claim 19, wherein the step of transducing glial cells further comprises transducing the glial cells ex vivo.

21. The method of claim 19, wherein the step of transducing glial cells further comprises transducing glial cells in vivo.

22. The method of claim 19, wherein the promoter is selected from the group consisting of human glial fibrillary acidic protein promoter (GFAP) and rat neuron specific enolase (NSE).

23. A method for treating amyotrophic lateral sclerosis (ALS) comprising:
delivering the nucleotide sequence encoding EAAT2 operably linked to a promoter functional to glial cells; and
expressing the EAAT2 protein a levels that ameliorate ALS.

24. The method of claim 23, wherein the step of delivering the nucleotide sequence encoding EAAT2 comprises delivering the nucleotide sequence encoding EAAT2 as a lipid entrapped sequence.

25. The method of claim 24, wherein the lipid is selected from the group consisting of egg phosphatidylcholine, dipalmitoylphosphatidylcholine, distearoylphosphatidylcholine, polylysine, protamine, sulfate and 3b -[N-(N′,N′ dimethylaminoethane) carbamoyl] cholesterol.

26. A method for treating epilepsy comprising:
transducing glial cells with a therapeutically effective amount of a recombinant adeno-associated virus (AAV) virion which has encapsidated therein a gene transfer vector comprising:
(i) a first AAV inverted terminal repeat sequence;
(ii) a nucleotide sequence encoding EAAT2 operably linked to a promoter functional in glial cells,
(iii) a second AAV inverted terminal repeat, wherein at least one of the elements (i) and (iii) comprises an AAV packaging signal; and
expressing the EAAT2 protein at levels that ameliorate epilepsy.

27. The method of claim 26, wherein the step of transducing glial cells further comprises transducing the glial cells ex vivo.

28. The method of claim 26, wherein the step of transducing glial cells further comprises transducing glial cells in vivo.

29. The method of claim 26, wherein the promoter is selected from the group consisting of human glial fibrillary acidic protein promoter (GFAP) and rat neuron specific enolase (NSE).
